(19) United States
(12) Patent Application Publication
(10) Pub. No.: US 2010/0262061 A1
(43) Pub. Date: Oct. 14, 2010

(54) Disposable self-contained wound protection device

(76) Inventors: Michael Thomas Fitzgerald, Huntington, WV (US); Jerry E. Hale, Kitts Hill, OH (US)

Correspondence Address: Michael T. Fitzgerald, 210 Baer Street, Huntington, WV, 25705, US

(21) Appl. No.: 12/384,909

(22) Filed: Apr. 11, 2009

(51) Int. Cl. A61F 13/00

(52) U.S. Cl. 602/54; 602/57

(57) ABSTRACT

A thin flat band (1) for wound protection of the type having at one end (24) a section of tape with protective backing (14) used to connect the band to its opposite end when formed into a loop (40). The band is made of a flexible material that can be easily formed into a loop. In addition, several rows of double sided tape (22) are affixed to the top (26) and bottom (28) edges of the band. The tape is notched to assist in the bending of the band. With the tape's protective backing (20) removed, one bottom rows of tape are used to attach the protector to the skin (32), the top rows can be used to attach additional protective layers of materials (48) to the protector.

### CROSS-REFERENCE TO RELATED APPLICATIONS

Not applicable.

### BACKGROUND

1. Field of Invention

This invention relates to wound protection. Specifically the prevention of contact of outside objects like clothing or bedding from various type of wounds, skin ailments, surgical stomas and sensitive areas of skin.

2. Description of Prior Art

Wound protection has taken many forms from simple dressings to disposable bandages. But there are times when an area of skin must be protected from contact and at the same time it must be readily accessible.

Wound protection started with simple coverings, then a gauze layer held together with strips of adhesive tape, then packaging to later become a single unit. An open wound may seep or run. Wound protection may become contaminated with exudate. Any material touching a wound may stick, cause infection, further injury or inflict pain.

There have been many versions since the design of the basic bandage. U.S. Pat. No. 5,718,695 to Keegan, McSpiritt and Rubinstein (Wound Protection Device) attempts protection of a wound while leaving access to the afflicted area though an opening in it's top. One limitation is that the unit come in a single size, is not adjustable, and limits the size of the area it can offer protect. It's height also limits the amount of protection it gives from clothing or bedding touching the wound and it offers no means to attach a top covering.

U.S. Pat. No. 11,409,364 to Aall (Wound Shield for Exudate Management) gives additional height in it's overall protection but comes in a single size, limiting the size or protected area. It is also not disposable nor self contained and requires additional items to make it ready for use.

U.S. Pat. No. 4,726,364 to George Spangler (Wound Protector with translucent cover) (US 1983) has the advantage as being a complete self contained unit. But the unit is not adjustable and is limited by the size of the opening in the foam pad. Also, the thin foam body does not give sufficient protection for raised wounds. And the permanently connected cover also limits the coverage options. This is not effective on raised wounds or wounds that need constant access to air.

### DISADVANTAGES TO PRIOR DESIGNS

Protection that completely covers some wounds won't let air in the area and it won't heal as quickly, medications can't be added as easily and wounds can't be monitored or cleaned as readily.
Any covering that touches the wound or sensitive areas can cause pain, discomfort or may adhere to the wound.
Protection that is comprised of several individual pieces are not as easy to construct and apply. They are also not cost effective in a professional setting.

### SUMMARY

In accordance with the present invention a wound protection device comprises of a thin plastic band having attached medical tape to adhere the protector to the skin and a adhesive pad to adjust the unit to the size necessary to protect a given area.

### OBJECTS & ADVANTAGES

Accordingly, besides the objects and advantages of the wound protect devices described above, many objects and advantages of the present invention are:

a) to provide a protective device that is affordable. A unit that can be a staple not only in the professional medical setting, but the home medical box. That can be sold in a pack containing several self-contained sealed ready to use units.
b) to provide a disposable protective device. In creating an affordable and easy to use unit, it also becomes disposable. This helps keep the area clean by simply replacing the unit if it becomes dirty or contaminated.
c) to provide a protective device that is easy and quick to apply.
d) to provide a protective device that is adjustable and can give protection to areas of varying sizes and shapes.
e) to provide a sterile protective device.
f) to provide a protective device that allows for optional top protection by adding a top layer to the supplied upper row of tape.
g) to provide a protective device that is self-contained. Having everything necessary on the unit to make it ready for immediate use.
h) to provide a protective device with an open top to allow access to the area for inspection, to apply medications or allow it to have open access to air.
i) to provide a protective device with extra height for protection of; raised wounds, infected areas, stitches, burns, boils, surgical stomas, inflammation.
j) to provide a device that allows one to wear clothing without it coming in contact with the wound.

### DRAWING FIGURES

In the drawings, closely related figures have the same number but different alphabetic suffixes.

FIGS. 1A to 1C show the individual components that make up a complete protective unit.

FIG. 2 shows the in side of an assembled unit with the sticky tape on the inside edge.

FIG. 3 shows the opposite side of the same assembled unit.

FIG. 4 shows a unit with notched tape in the ready position for use.

FIG. 5 shows a protective unit after it has been formed into a loop and has been secured by sticky tape pad on the opposite end of the band.

FIG. 6 shows the unit with the tape sections deployed for use.

FIG. 7 shows the unit with additional covering attached via the top tape section.

FIG. 8A to 8B shows the components that make up the packaging for the unit

FIG. 9 shows a package with a logo example

FIG. 10 shows both a see-through and cutaway view of an assembled package with a complete protective unit in place.

### REFERENCE NUMERALS IN DRAWINGS

10 protector band
12 rounded edge
14 sticky tape pad
16 peel-off protective layer
18 medical tape strip
20 peel-off protective layer
22 notch
24 location of sticky tape pad
26 top strip of medical tape
28 bottom strip of medical tape
30 top strip of tape in ready position
32 bottom strip of tape in ready position
40 band ends connected
42 lower tape affixed to skin
44 band formed into loop
46 top strip of tape in ready position
48 additional covering
50 covering attached to top
64 package back section
62 rectangle of adhesive
64 package front section
66 logo imprint example
58 package opening area
70 unit in packaging

### DESCRIPTION

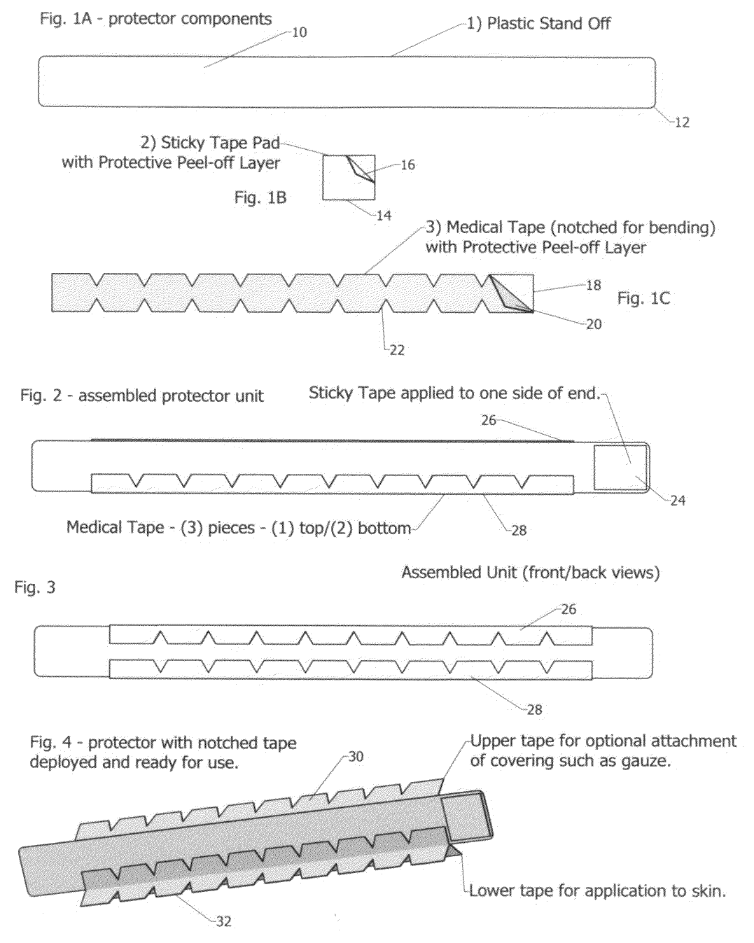

### FIGS. 1A-1C

### Components

The components that make up the completed protective unit are shown in FIGS. 1A-1C. The main band is a slender plastic band (10) with rounded corners (12) as to not dig into the skin. A sticky pad (14) with peel-off protective backing (16) is used to adhere the band end to the opposite end when the band is initially formed into a loop.

Three strips of medical tape (18), also with a peel-off protective backing (20) are affixed to the band (10). These strips of tape are notched (22) to facilitate bending when the initial loop is formed.

### FIGS. 2 and 3

### Preferred Embodiment

On one side of one end of the band is the sticky pad located (24). The three strips of notched tape are folded half back against themselves with one half of the strip affixed to the band and the remaining portion keeping it's peel-off backing in place to be removed when later needed.

One tape strip is affixed in this manner along the top of the band (26) on the side opposite of the sticky pad (24). The other two strips are affixed along the bottom of the band (28).

### FIG. 4

### Preparation for Use

The preparation of the notched tape strips is detailed in FIG. 4. In each case the peel-off protective backing of each strip is first removed and the folded sections are pulled up away from the band. As deployed in (30) the top band is now ready to accept an optional top covering. And as detailed in (32) the lower tape sections are ready to affix to the skin.

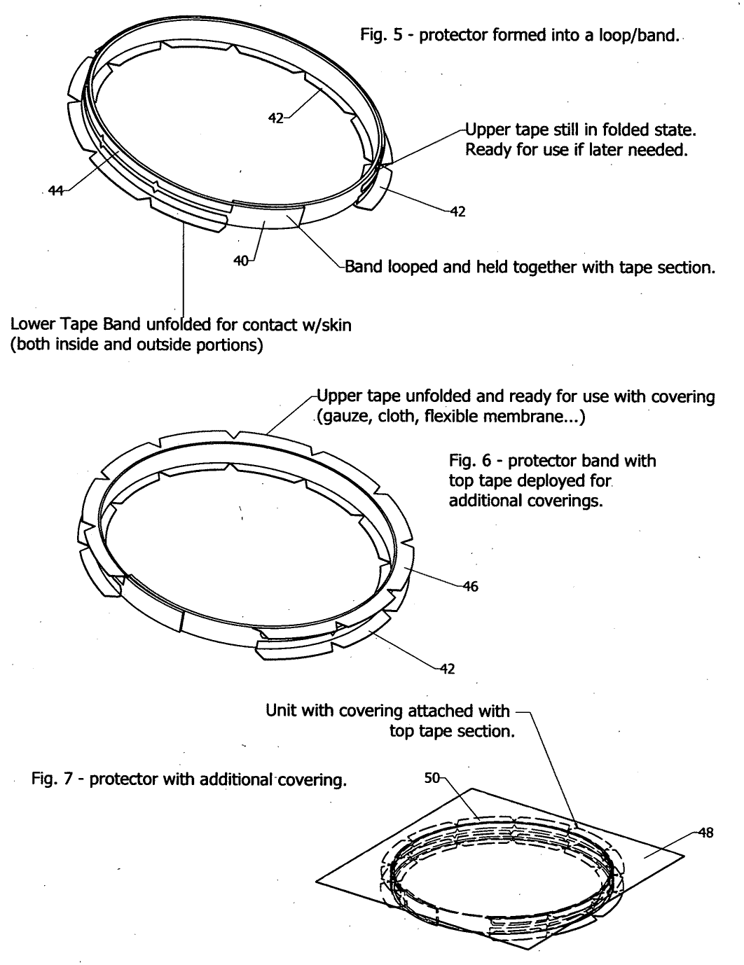

### FIG. 5

### Band in Use

The sticky tape pad with peel-off protective backing removed is affixed to the opposite end of the band (40). The lower tape bands have had their backing removed and are folded down away from the side of the band (42). When the unit is placed on the skin surrounding the affected area, it is these two sections of tape that will hold the band to the skin.

### FIGS. 6 and 7

### Top Tape Row Deployment

If an additional top layer of covering is needed, the top row of tape is prepared (backing removed and pulled away from the unit) and the covering (48) (gauze, breathable membrane . . . ) is place across the exposed top row of tape (50).

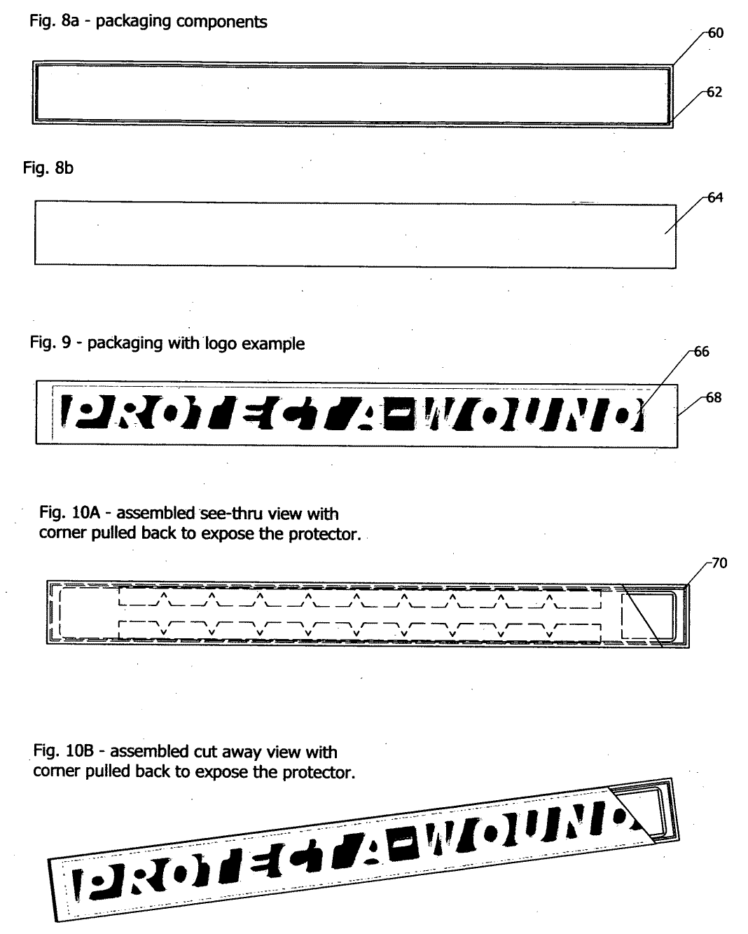

### FIGS. 8A and 8B

### Packaging

The preferred packaging for the individual unit is to enclose the completed protective unit between two sections of paper (60) and (64) which are sealed with a strip of adhesive. One end of the package has a small segment of the ends unattached to assist in the opening of the package.

### ADVANTAGES

From the drawings above, a number of advantages of our disposable wound protector become evident:

a) Bands of varying heights can be included in a set giving protection to raised wounds or inflamed areas of skin.

b) Individual packaging allows each unit to carry the product logo and basic operational information.

c) The unit quickly becomes more cost effective than other makeshift devices constructed from several separate components.

### OPERATION

### FIGS. 1, 2, 5, 7, 9, 10

The manner of use of the Wound Protector is similar to that of a packaged bandage. The unit is contained in a sterile peel apart wrapper (50). The wrapper ends are separated and the unit is removed (40). If a longer final assembly will be needed, additional units may be removed from their packages.

The unit is positioned as such, the side with the square of sticky tape in the center is the in side (24). There are two rows of notched tape on the top and bottom of the plastic band. The side with the two strips is the bottom (28), the other with the single, the top (26). The top side with no tape is the inside.

Remove the protective backing from the notched tape on the bottom inside strip (20). Next remove the protective backing from the sticky tape on the end of the plastic strip (16). If other strips will be joined to create a longer final assembly, adhere the exposed tape to the back outside end of another plastic band to create a longer final unit.

If using only one strip, bend the strip around and stick the tape to the outside edge of the rear of the strip to complete a loop (40). If the loop is smaller than the complete strip length, any extra plastic may be cut away from the back of the unit.

Place the looped protective around the wound area, being careful not to make contact with the wound or any sensitive skin. Make sure to leave enough space between the edge of the protector and the wound to adhere the inside portion of the adhesive tape.

Push down on the tape section on the inside of the ring (42), adhering the tape to the skin. Next remove the protective strip from the outside layer of tape and press the outside row of tape down to the skin (42). The unit is now in position to protect the wound.

If additional coverage is needed, removed the protective tape from the top row of tape (46) and lay appropriate covering across the top of the unit (48). Press the section of tape upward to make contact with the protective material (50). The protective layer is now be held in place.

### CONCLUSIONS, RAMIFICATIONS AND SCOPE

Accordingly, one can see that the wound protection invention outlined above can quickly, affordably and conveniently be used to protect from contact wounds, sensitive areas of skin and surgical stomas. The wound protection invention provides this in a complete, inexpensive, sterile and ready to use package.

Furthermore, the wound protector has the additional advantages in that:

It provides a protective device that can designed in different heights and overall length.

It provides a protective device that can be easily applied by one person. Many applications would not need any assistance to apply unit to most wounds (depending on location). This means it can be applied at any time.

It provides a device that lets one sleep or rest without clothing or bedding coming in contact with wounds or sensitive areas.

It provides a protective device that is affordable. A unit that can be a staple not only in the professional medical setting, but the home medical box. That can be sold in a pack containing several self-contained sealed ready to use units. A unit that ships in a standard bandage like packaging.

It provides a disposable protective device. In creating an affordable and easy to use unit, it also becomes disposable. This helps keep the area clean by simply replacing the unit if it becomes dirty or contaminated.

It provides a protective device that is quick and easy to apply.

It provides a protective device that is adjustable and can give protection to areas of varying sizes and shapes.

It provides a sterile protective device.

It provides a protective device that allows for optional top protection by adding a top layer.

It provides a protective device that is self-contained. Having everything necessary on the unit to make it ready for immediate use.

It provides a protective device with an open top to allow access to the area for inspection, to apply medications or to let it have open access to air.

I claim:

1. A flexible band of the type comprising of a flat thin body of rectangle shape.

2. the band of said body of material is composed of plastic

3. the band of said body has rounded corners.

4. the band of claim 1 wherein said body has several strips of adhesive tape running along its length.

5. the strips of said adhesive tape have notches running along both sides of their length.

6. the strips of said tape are folded in half against themselves, one half attached partway down the band.

7. the exposed sections of the said adhesive strips have a protective peel off backing.

8. the band of claim 1 wherein said body has a square of adhesive tape attached near the end on one side of said band,

9. the exposed side of said adhesive square has a protective peel off backing.
